(19) United States
(12) Patent Application Publication
(10) Pub. No.: US 2006/0063742 A1
(43) Pub. Date: Mar. 23, 2006

(54) Method of treatment for or protection against lymphedema

(76) Inventor: Frederick H. Hausheer, Fair Oaks Ranch, TX (US)

Correspondence Address: Thomas J. Dodd, Senior Patent Counsel;BioNumerik Pharmaceuticals, Inc., Suite 1250, 8122 Datapoint Drive, San Antonio, TX, 78229, US

(21) Appl. No.: 10/945,754

(22) Filed: Sep. 21, 2004

(51) Int. Cl. A61K 31/66; A61K 31/185

(52) U.S. Cl. 514/114; 514/553; 514/126

(57) ABSTRACT

A method of reducing the risks of lymphedema, particularly secondary lymphedema associated with surgery or radiotherapy is disclosed. The method of this invention includes administering effective amounts of specific sulfur-containing drug agents according to Formula I herein to a patient at risk of developing lymphedema.

### FIELD OF THE INVENTION

This invention relates to a method of treating or protecting against lymphedema. The method is especially useful as a prophylaxis against lymphedema for patients undergoing surgery or radiation therapy for cancer or other diseases where peripheral lymph nodes must be removed.

### BACKGROUND OF THE INVENTION

Lymphedema is a condition that refers to edema from accumulation of lymph secondary to the obstruction of its flow. Lymphedema is characterized by generalized painful swelling in the affected area.

The most common type of primary lymphedema is simple congenital lymphedema, which is not familial, and is present at birth. Milroy's Disease and Noonan's Syndrome are inherited autosomal dominant forms of primary lymphedema, seen in about 15 percent of cases. Primary lymphedema is most commonly present in the legs, but may manifest in any area of the body.

Primary lymphedema is more commonly found in women. Most cases manifest at birth or become apparent before age 40. Secondary effects of the condition may include yellowing of the nails and recurrent pleural effusion. A familial syndrome consisting of recurrent intrahepatic cholestasis and lymphedema is thought to be caused by defective hepatic lymphatic vessels as well as vessels located in the extremities. Pathologically, primary lymphedema results from either the absence of lymphatic vessels in the affected area, or from hypoplasia thereof.

Secondary lymphedema most commonly results from trauma. It normally manifests in the upper arm, and manifests often after surgical removal of lymph nodes and from fibrosis following radiation and/or surgery. Most commonly, patients who have undergone surgery and/or radiation therapy for breast cancer or lymphoma will develop secondary lymphedema in the arm adjacent to the area of the removed lymph vessels. Secondary lymphedema may also be brought on by various infections. Pathologically, in secondary lymphedema there are often found numerous small lymphatics, together with tortuous and sometimes greatly enlarged varicose lymphatic vessels.

Lymphedema typically begins gradually with an enlargement of the involved limb often without other symptoms. The swollen extremity is most often soft and pitting and the swelling usually subsides at night. After a time, the skin thickens and cannot be raised into a fold, and the edema becomes more persistent. Superimposed lymphangitis and cellulitis may develop and in longstanding cases the patient may develop a lymphangiosarcoma.

Primary lymphedema is usually a slow and progressive disorder and not easily amenable to treatment. Secondary lymphedema treatment depends upon the underlying cause. Currently, when the secondary lymphedema is caused by infection, the lymphedema can be managed by treatment with antibiotics.

Treatment of primary lymphedema generally involves measures such as elevation of the limb, use of elastic stockings, administration of diuretics, and in more advanced cases administration of benzopyrone anticoagulant agents, such as warfarin. In severe cases, surgery to remove the subcutaneous tissue and induce new lymph vessel formation has been tried with some success. All of the prior treatments, even if successful, carry risks, particularly the administration of drug agents, all of which carry significant risks of adverse effects.

Mesna (sodium 2-mercaptoethene sulfonate) and dimesna (disodium 2,2′-dithiobis ethane sulfonate) are known therapeutic compounds that have heretofore demonstrated a wide variety of therapeutic uses. Both mesna and dimesna have been shown to be effective protective agents against certain specific types of toxicity associated with the administration of cytotoxic drugs used to treat patients for various types of cancer.

In particular, mesna has been used with some success in mitigating the toxic effects of cytotoxic agents such as ifosfamide, oxazaphosphorine, melphalane, cyclophosphamide, trofosfamide, sulfosfamide, chlorambucil, busulfan, triethylene thiophosphamide, triaziquone, and others, as disclosed in U.S. Pat. No. 4,220,660, issued Sep. 2, 1980.

The near absence of toxicity of dimesna further underscores the usefulness of this compound, as large doses can be given to a patient without increasing the risk of adverse effects from the protective agent itself.

Further, pharmacological profiles of each compound indicate that, if proper conditions are maintained, mesna and dimesna do not prematurely inactivate primary therapeutic drugs to a significant degree. Thus, neither compound will significantly reduce activity of the chemotherapeutic agent, and in many cases, act to potentiate the effect of the main drug on targeted cancer cells.

The molecular structures of both mesna and dimesna are shown below as Structure I and Structure II respectively.
HS—CH₂—CH₂—SO₃Na (I)
NaSO₃—CH₂—CH₂—S—S—CH₂—CH₂—SO₃Na (II)

As shown, dimesna is a dimer of mesna, with the optimum conditions for oxidation occurring in the slightly basic (pH ^{˜}7.3), oxygen rich environment found in blood plasma. In mildly acidic, low oxygen conditions, in the presence of a reducing agent such as glutathione reductase, conditions prevalent in the kidneys, the primary constituent is mesna.

Mesna acts as a protective agent for a number of cytotoxic agents by substituting a nontoxic sulfhydryl moiety for a toxic hydroxy (or aquo) moiety. This action is particularly evidenced in the coadministration of mesna and oxazaphosphorine, and in the administration of dimesna along with certain platinum agents and/or taxanes.

Dimesna, as well as some analogues, have excellent toxicity profiles in mammalian species. In fact, dimesna has been administered intravenously to mice and dogs in doses higher than the accepted oral LD₅₀ for common table salt (3750 mg/kg), with no adverse effects. Dimesna has also been administered to humans in doses exceeding 40 g/m², with no adverse effects.

Mesna, and other analogues with free thiol moieties, constitute the more physiologically active form of the two types of compounds described in this specification. These compounds manifest their activity by providing free thiol moieties for terminal substitution at locations where a terminal leaving group of appropriate configuration, usually a hydroxy, aquo or superoxide is located. Mesna also tends to form conjugates with naturally occurring biochemicals that contain a free thiol moiety, such as cysteine, glutathione, homocysteine, and others.

Dimesna and other disulfides can be activated intracellularly by glutathione reductase, a ubiquitous enzyme, thereby generating high concentrations of intracellular free thiols. These free thiols act to scavenge the free radicals and other nucleophilic compounds often responsible for causing cell damage.

This profile is especially significant in explaining the success of dimesna in controlling and mitigating the toxic effects of platinum complex antitumor drugs. The mechanism for action in the case of cisplatin (cis-diammine dichloro platinum) is explained in U.S. Pat. No. 5,789,000, which is incorporated herein by reference.

Mesna, dimesna, and analogues of these compounds have been the subject of several prior pharmaceutical uses described in the literature and in prior patents, both in the United States and around the world. In addition to the cytotoxic agent protection uses, one or more of these compounds have proven effective, in vitro, against a multiplicity of biological targets, and have been effective, in vivo, in the treatment of sickle cell disease, radiation exposure, chemical agent exposure, and other uses.

Mesna, dimesna, and analogues thereof are synthesized from commonly available starting materials, using acceptable routes well known in the art. One such method involves the two-step, single pot synthetic process for making dimesna and like compounds of the following formula:
R₁—S—R₂;
wherein:

R₁ is hydrogen, X-lower alkyl, or X-lower alkyl-R₃;

R₂ is -lower alkyl-R₄;

R₃ and R₄ are each individually SO₃M or PO₃M₂;

X is absent or X is sulfur; and

M is an alkali metal.

The process essentially involves a two-step single pot synthetic process, which results in the conversion of an alkenyl sulfonate salt or acid to the desired formula I compound. The process in the case of mesna is a single step process that converts the alkenyl sulfonate salt to mesna or a mesna derivative by reacting with an alkali metal sulfide or with hydrogen sulfide.

If the desired end product is dimesna or a dimesna analogue, a two-step single pot process is involved. Step 1 is as described above. Step 2 of the process is performed in the same reaction vessel as Step 1 without the need to purify or isolate the mesna formed during that step. Step 2 includes the introduction of oxygen gas into the vessel, along with an increase in pressure and temperature above ambient values, at least 20 pounds per square inch (psi) and at least 60° C. Dimesna or a derivative thereof is formed in essentially quantitative yield.

Other processes, well known and documented in the prior art, may be employed to make either mesna or dimesna, or derivatives and analogues thereof.

### SUMMARY OF THE INVENTION

The method of this invention includes the administration of an effective amount of a formula I compound to a patient suffering from lymphedema or at risk of developing lymphedema.
R₁—S—R₂—R₃ (I)
wherein

R₁ is hydrogen, lower alkyl or —S—R₂—R₃;

R₂ is lower alkylene, optionally substituted by one or more hydroxy, alkoxy, mercapto, nitro or amino moieties for a corresponding hydrogen atom; and

R₃ is sulfonate or phosphonate; and

pharmaceutically acceptable salts thereof.

### DESCRIPTION OF THE PREFERRED EMBODIMENT

The preferred embodiment herein described is not intended to be exhaustive or to limit the invention to the precise form disclosed. It is chosen and described to explain the principles of the invention and its application and practical use to enable others skilled in the art to best follow its teachings.

The method of this invention has application in the medical field, particularly in the treatment and/or prevention of lymphedema. As stated above, the method involves the administration of an effective amount of a formula I compound to a patient suffering from lymphedema or to a patient at risk from developing lymphedema due to a forthcoming surgical procedure or radiation therapy.

Administration of the formula I compound is through one of several accepted routes, such as oral, topical or parenteral. In oral administration, the formula I compound is contained in a swallowable form, such as a tablet, capsule, caplet, lozenge, soluble powder, or other form suitable for oral administration. For topical administration, the formula I compound is mixed with suitable pharmaceutically acceptable excipients to form a lotion or cream or other topical application form. For intravenous or subcutaneous administration, the formula I compound is dissolved or suspended in a pharmaceutically acceptable solvent for administration.

Risk of secondary lymphedema is highest among patients undergoing surgery that requires removal of lymph nodes, namely surgeries for breast cancer or lymphoma, and in patients undergoing radiation therapy.

Timing of the administration of the formula I compound depends on the amount of the formula I compound to be administered, the preferred route of administration, whether the formula I compound is used as a prophylaxis or as treatment, and other factors common or perhaps unique to each individual situation. When used for prophylactic purposes, the formula I compound is preferably administered prior to any surgery or radiation therapy.

Preferred timing for administration of the formula I compound in the prophylaxis of secondary lymphedema is from five minutes to one hour prior to surgery or radiation therapy. The formula I compound is preferably administered thereafter to maximize the concentrations of the formula I compound during the time frame when the patient faces the highest risk of developing lymphedema.

The effective amount of formula I compound to be administered is defined as that amount which safely and effectively reduces the risk of lymphedema in susceptible patients, or the amount that effectively treats a patient suffering from lymphedema. Exact amounts will vary from patient-to-patient, as will results and the route of administration and timing will also affect the preferred dosage. Effective oral and parenteral doses may range from as little as 0.1 g/m² up to 80.0 g/m² or higher. Preferred dosage amounts are from 4.0 g/m² to 42.0 g/m². Preferred timing of administration is from five minutes to about one hour prior to commencement of surgery or radiation for prophylaxis of secondary lymphedema. For treatment of primary or secondary lymphedema, the formula I compound is preferably administered as soon as possible following diagnosis, with follow-up doses administered until positive results are obtained.

The formula I compound may also be administered subsequent to the completion of surgery or radiation therapy. Subsequent doses reduce the risk of any delayed symptoms from the surgery or radiation therapy that may not manifest soon thereafter. Subsequent doses may be self-administered by the patient as needed if swelling occurs that would indicate the onset of lymphedema. These subsequent doses may take place at regular intervals following the surgery or radiation therapy at two to twelve hour intervals, most preferably four hours to six hours between doses.

Topical dosage forms are typically more complex, both in formulation and measurement of dose. A number of factors influence dose amounts, including but not limited to the concentration of active ingredient; the drug's absorption rate through the skin into surrounding tissues as well as into the bloodstream; effects of any additives and excipients in the formulation; and others. Due to the ability to apply the drug directly to the target area, drug metabolism and distribution are not as critical as with other dosage forms, and topical dosage amounts are often lower than oral or parenteral dosages of the same drug.

The formula I compound is prepared for administration by commonly known synthetic processes, such as the processes taught in U.S. Pat. No. 5,808,140, incorporated herein by reference. After sterilization, the formula I compound is formulated for administration to the patient, with the preferred formulation dependant on the form of administration.

For intravenous or subcutaneous or subdermal or intradermal administration, the formula I compound is dissolved in a suitable solvent, preferably water. Suitable excipients may also be added to the formulation, as described in U.S. Pat. Nos. 5,789,000; 5,919,816; and 5,866,169, which are incorporated herein by reference.

For oral administration, the formula I compound may be combined with pharmaceutically acceptable fillers and then administered as tablets, caplets, or other swallowable form. Alternatively, the formula I compound may be dissolved or suspended in a pharmaceutically acceptable solvent and encapsulated in a swallowable carrier such as a capsule, a gel cap, or other form, or the formula I compound may be dissolved and then administered as a solution or suspension.

For topical administration, the formula I compound is mixed with pharmaceutically acceptable excipients to produce an elegant formulation designed to deliver the formula I compound to the tissue site surrounding the injection area. The formula I compound is preferably dissolved or suspended in a solvent vehicle, most preferably purified water, prior to addition of the excipients. The pharmaceutically acceptable excipients used to create the formulation may include one or more plasticizers, emulsifiers, emollients, pH adjusters, skin penetration enhancers, surfactants, thickening or thinning agents depending on the desired viscosity and applicability of the formulation, and other ingredients as desired.

Administration of the formula I compound is preferably according to one of the following examples, which are illustrative only and not to be considered as limiting the invention to the described details.

### EXAMPLE 1

### Intravenous Administration

A patient about to undergo radical mastectomy and lymph node removal surgery is administered an intravenous dose of 20 g/m² of a sterile solution of disodium 2,2′-dithiobis ethane sulfonate over 15 minutes by slow drip infusion. 15 minutes after the infusion is completed, the surgical procedure is commenced. Over the course of the following 7 days, the patient is given additional infusions of 20 g/m² of disodium 2,2′-dithiobis ethane sulfonate every 4 hours, and the patient's progress is monitored for any signs of lymphedema.

### EXAMPLE 2

### Oral Administration

A patient about to undergo radiation therapy is given an oral dose of 20 g/m² of disodium 2,2′-dithiobis ethane sulfonate in a swallowable carrier. 15 to 45 minutes after taking the dose, the patient begins the radiotherapy. The patient is then given, or self-administers additional oral doses every 4 hours, with progress monitoring for lymphedema symptoms determining how long the patient is given additional doses of drug.

### EXAMPLE 3

### Topical Administration

A lumpectomy and lymph node removal surgical procedure is completed on a patient, who then has a pharmaceutically elegant topical formulation of disodium 2,2′-dithiobis ethane sulfonate applied to the area of the removed nodes and the surrounding areas. Further applications of the topical formulation may be applied as desired or deemed necessary by the attending health care professional, and the patient's progress is monitored for symptoms of lymphedema.

### EXAMPLE 4

### Direct Injection Administration

A patient about to undergo a surgical procedure for removal of lymph nodes is administered a dose of a sterile solution or suspension of 1%-75% w/w disodium 2,2′-dithiobis ethane sulfonate by direct tissue injection (subcutaneous, subdermal and/or intradermal) approximately 15 to 45 minutes prior to beginning the procedure.

It should be further noted that combinations of the recited methods of administration may be practiced according to the teachings of this invention. For example, a patient may receive a pretreatment dose (by any accepted route of administration) of formula I compound prior to beginning surgery or radiotherapy, and then be given oral dosages or topical formulation to take home, along with instructions to self-administer or apply the doses following the surgery or radiotherapy at regular intervals thereafter. Allowing the patient to self-administer subsequent doses aids in convenience and independence for the patient and builds self-esteem, often an important psychological factor for patients who require surgery and/or radiotherapy that places them at risk of developing a secondary lymphedema.

The above description has been presented for illustrative purposes to enable those skilled in the art to understand its teachings, and is not to be considered as limiting the scope of the invention to the precise details herein recited, which scope is defined in the foregoing claims.

I claim:

1. A method for reducing or preventing lymphedema in a patient, said method comprising administering to said patient an effective amount of a formula I compound:
R₁—S—R₂—R₃ (I)
wherein
R₁ is hydrogen, lower alkyl or —S—R₂—R₃;
R₂ is lower alkylene, optionally substituted by one or more hydroxy, alkoxy, mercapto, nitro or amino moieties for a corresponding hydrogen atom; and
R₃ is sulfonate or phosphonate; and
pharmaceutically acceptable salts thereof.

2. The method of claim 1 wherein the effective amount of the formula I compound is from 0.1 g/m² to 80.0 g/m².

3. The method of claim 1 wherein the risk of lymphedema to the patient is from surgery or radiotherapy.

4. The method of claim 3 wherein the effective amount of the formula I compound is administered from 5 minutes to one hour prior to beginning the surgery or radiotherapy.

5. The method of claim 1 wherein R₂ is ethylene, R₃ is sulfonate and R₁ is —S—R₂R₃.

6. The method of claim 4 wherein the effective amount is from 4.0 g/m² to 42.0 g-m².

7. The method of claim 4 wherein the effective amount is administered from 25 minutes to 30 minutes prior to beginning the surgery or radiotherapy.

8. The method of claim 4 wherein the effective amount is administered at intervals subsequent to the completion of the surgery or radiotherapy.

9. The method of claim 8 wherein the intervals are every two to twelve hours following completion of the surgery or radiotherapy.

10. The method of claim 4 wherein the formula I compound is administered intravenously.

11. The method of claim 4 wherein the formula I compound is administered subcutaneously.

12. The method of claim 4 wherein the formula I compound is administered topically.
